(19) United States
(12) Patent Application Publication
(10) Pub. No.: US 2021/0220618 A1
(43) Pub. Date: Jul. 22, 2021

(54) CATHETER ASSEMBLY

(71) Applicant: TERUMO KABUSHIKI KAISHA, Tokyo (JP)

(72) Inventor: Masahiro ISHIDA, Hadano-shi (JP)

(73) Assignee: TERUMO KABUSHIKI KAISHA, Tokyo (JP)

(21) Appl. No.: 17/199,843

(22) Filed: Mar. 12, 2021

Related U.S. Application Data
(63) Continuation of application No. PCT/JP2019/032479, filed on Aug. 20, 2019.

(30) Foreign Application Priority Data
Sep. 19, 2018 (JP) 2018-174977

(51) Int. Cl. A61M 25/06; A61M 25/00

(52) U.S. Cl. CPC A61M 25/0606; A61M 25/0074; A61M 25/0693

(57) ABSTRACT

A catheter assembly includes a catheter and an inner needle. The catheter has a catheter body and a flexible portion that is more flexible than the catheter body. The inner needle is provided with a backcut portion. The relationship between a height of the backcut portion and a length from a distal-most portion of the catheter to a distal-most portion of the catheter body satisfies a formula A>0.03B.

### CROSS-REFERENCE TO RELATED APPLICATIONS

This application is a bypass continuation of PCT Application No. PCT/JP2019/032479, filed on Aug. 20, 2019, which claims priority to Japanese Application No. 2018-174977, filed on Sep. 19, 2018. The contents of these applications are hereby incorporated by reference in their entireties.

### BACKGROUND

The present disclosure relates to a catheter assembly configured to be punctured and indwell in a blood vessel when performing an infusion or the like to a patient, for example.

Conventionally, a catheter assembly used when performing an infusion or the like to a patient has been known. This kind of the catheter assembly includes a hollow catheter, a catheter hub fixed to a proximal end of the catheter, a hollow inner needle that is inserted into the catheter and has a sharp needle tip at a distal end, and a needle hub fixed to a proximal end of the inner needle (for example, see JP 2008-43445 A). When using a catheter assembly, skin and a blood vessel of a living body are punctured the distal ends of the inner needle and the catheter, and the catheter is then advanced with respect to the inner needle so that the catheter is inserted into the blood vessel by a predetermined length.

### SUMMARY

In a conventional catheter assembly having an inner needle and a catheter, a catheter distal end is sometimes caught by a blood vessel back wall (a blood vessel wall opposing a puncture spot) when a puncture angle, which is an angle between a central axis of the blood vessel to be punctured and a central axis of the inner needle with which the puncture is performed, is large. As a result, the catheter is hardly inserted into a blood vessel or the blood vessel wall is damaged by the catheter distal end.

Certain embodiments of the present invention have been developed in consideration of such a problem, and one object thereof is to provide a catheter assembly capable of inhibiting a catheter distal end from being caught by a blood vessel back wall at the time of advancing a catheter to a blood vessel even when a puncture angle, which is the angle between a central axis of the blood vessel to be punctured and a central axis of an inner needle with which the puncture is performed, is large.

According to one embodiment, a catheter assembly includes: a catheter and an inner needle inserted through the catheter. The catheter has a catheter body and a flexible portion that is provided at a distal end of the catheter body, includes a distal-most portion of the catheter, and is more flexible than the catheter body. The inner needle is provided with a backcut portion. The relationship between a height A of the backcut portion and a length B from the distal-most portion of the catheter to a distal-most portion of the catheter body satisfies a formula of A>0.03B.

According to certain embodiments of the catheter assembly, the flexible portion, which is more flexible than the catheter body, is provided at the distal portion of the catheter body, and thus, it is possible to prevent the distal end of the catheter from being caught by the blood vessel back wall even when the puncture angle, which is an angle between a central axis of the blood vessel to be punctured and a central axis of the inner needle with which the puncture is performed, is large. Accordingly, it is possible to inhibit the catheter from being difficult to insert into a blood vessel or to prevent a blood vessel wall from being damaged by the catheter distal end. Because the relationship between the height A of the backcut portion and the length B from the distal-most portion of the catheter to the distal-most portion of the catheter body satisfies the formula of A>0.03B, the height of the backcut portion is appropriately high so that it is possible to prevent an inner surface of the catheter from being punctured with the needle tip.

### BRIEF DESCRIPTION OF DRAWINGS

FIG. 1 is a perspective view of a catheter assembly according to an embodiment of the present invention;

FIG. 2A is a cross-sectional view of a distal portion of the catheter assembly;

FIG. 2B is an explanatory view of a shape of a distal portion of an inner needle; and

FIG. 3 is an explanatory view of a function of the catheter assembly.

### DETAILED DESCRIPTION

Hereinafter, preferred embodiments of a catheter assembly according to the present invention will be described with reference to the accompanying drawings.

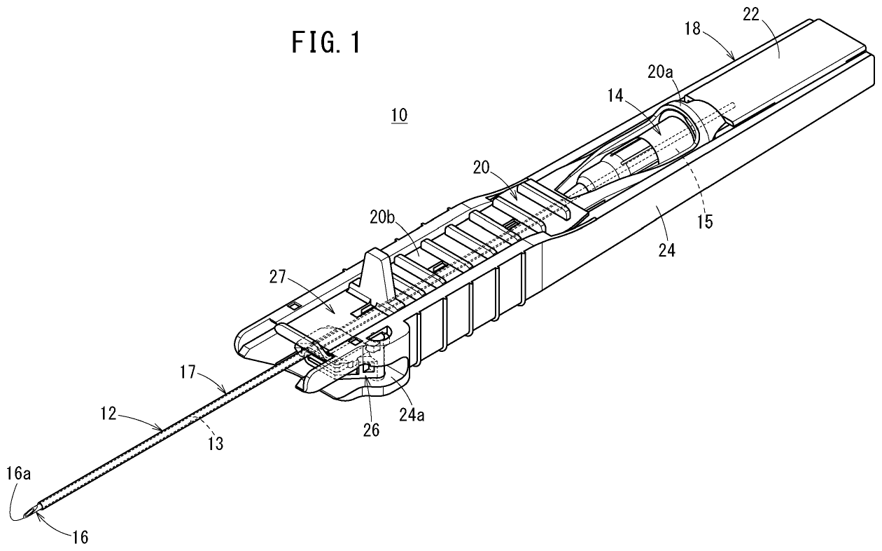

The catheter assembly 10, whose initial state is illustrated in FIG. 1, is applied when performing an infusion, a blood transfusion, and the like to a patient (living body), and forms an introduction portion for a medicinal liquid or the like by being punctured the patient's body to indwell. The catheter assembly 10 may be configured as a catheter having a longer length (for example, a central venous catheter, a PICC, a mid-line catheter, and the like) than a peripheral venous catheter. Alternatively, the catheter assembly 10 may be configured as a peripheral venous catheter. In addition, the catheter assembly 10 is not limited to the venous catheter, and may be configured as an arterial catheter such as a peripheral arterial catheter.

As illustrated in FIG. 1, the catheter assembly 10 includes a catheter 12, a catheter hub 14 fixedly holding the catheter 12, a hollow inner needle 16 removably inserted into the catheter 12, a needle hub 18 fixedly holding the inner needle 16, and a catheter operation member 20 mounted to the catheter hub 14. The inner needle 16 may be a solid needle.

The catheter assembly 10 forms a multi-tube structure (multi-tube portion) in which the catheter 12 and the inner needle 16 are sequentially stacked in an initial state before use.

The catheter 12 is flexible and has a lumen 13 that extends therethrough. The lumen 13 is formed to have a diameter capable of accommodating the inner needle 16 and capable of causing a medicinal liquid, blood, or the like to flow. A distal end of the catheter 12 is reduced in diameter in order to decrease a puncture resistance, and an inner surface of the catheter 12 is in close contact with an outer surface of the inner needle 16 at such a reduced diameter portion in the initial state of the catheter assembly 10. A length of the catheter 12 is not particularly limited but can be appropriately designed according to use and various conditions, and is set to, for example, about 14 to 500 mm, about 30 to 400 mm, or about 76 to 200 mm.

A proximal portion of the catheter 12 is fixed to a distal portion inside the catheter hub 14. The catheter 12 and the catheter hub 14 form a catheter member 17.

The catheter hub 14 is exposed on the patient's skin in a state in which the catheter 12 has been inserted into a blood vessel, and indwelled together with the catheter 12 by being affixed with a tape or the like. The catheter hub 14 is formed in a tubular shape that is tapered in a distal direction.

A constituent material of the catheter hub 14 is not particularly limited, but a thermoplastic resin, such as polypropylene, polycarbonate, polyamide, polysulfone, polyarylate, a methacrylate-butylene-styrene copolymer, and polyurethane may be preferably used.

A hollow portion 15 that communicates with the lumen 13 of the catheter 12 and through that an infusion solution can flow is provided inside the catheter hub 14. A hemostatic valve, a plug, or the like (not illustrated) may be accommodated inside the hollow portion 15 in order to prevent back-flow of blood at the time of puncture with the inner needle 16 and to allow infusion along with insertion of a connector of an infusion tube.

The inner needle 16 is configured as a hollow tube having rigidity that enables puncture of a skin of a living body, and is arranged to penetrate through the lumen 13 of the catheter 12 and the hollow portion 15 of the catheter hub 14. The inner needle 16 is formed to have a total length longer than that of the catheter 12, and a sharp needle tip 16a is provided at a distal end thereof. A lumen penetrating in an axial direction of the inner needle 16 is provided inside the inner needle 16, and this lumen communicates with a distal opening of the inner needle 16.

Examples of a constituent material of the inner needle 16 include a metal material such as stainless steel, aluminum or an aluminum alloy, and titanium or a titanium alloy, a hard resin, ceramics, and the like.

The needle hub 18 has a needle holding member 22 fixed to a proximal portion of the inner needle 16, and a housing 24 to which the needle holding member 22 is fixed and that extends along the inner needle 16 and the catheter 12. In the initial state of the catheter assembly 10, the housing 24 houses a part of the multi-tube structure, the catheter hub 14, and the catheter operation member 20. Resin materials forming the needle holding member 22 and the housing 24 are not particularly limited. However, for example, the materials exemplified for the catheter hub 14 can be appropriately selected. Incidentally, the needle holding member 22 and the housing 24 may be monolithically formed.

When the needle hub 18 is moved to a proximal direction with respect to the catheter 12, the inner needle 16 is also moved in the proximal direction with respect to the catheter 12 along with the movement of the needle hub 18 because the needle hub 18 holds the inner needle 16 at the needle holding member 22.

The catheter operation member 20 is attached to the catheter hub 14. Thus, when the catheter operation member 20 is advanced relative to the needle hub 18, the catheter member 17 is advanced relative to the inner needle 16. The catheter operation member 20 has a hub mounting portion 20a detachably mounted on the catheter hub 14, and an operation plate portion 20b extending from the hub mounting portion 20a along the catheter 12 in the distal direction. Incidentally, the catheter operation member 20 is not necessarily provided in the catheter assembly 10.

The catheter assembly 10 is provided with a support member 26 on the distal side of the housing 24 in order to support a lower side of the catheter 12 held by the catheter operation member 20. The support member 26 is rotatably attached to an arrangement concave portion 24a provided at a distal portion of the housing 24. A distal portion of the catheter operation member 20 and the support member 26 constitute a deflection suppressing mechanism 27.

When the skin is punctured with the inner needle 16 and the catheter 12, the distal portion of the catheter operation member 20 supports the catheter 12 from above and the support member 26 supports the catheter 12 from below, and thus, deflection of the catheter 12 and the inner needle 16 is suppressed. When the catheter operation member 20 is removed from the housing 24, the support member 26 is rotated toward an outer side of the housing 24 by being pushed by the hub mounting portion 20a, and thus, the catheter hub 14 can be withdrawn from the housing 24 in the distal direction. Incidentally, the support member 26 is not necessarily provided.

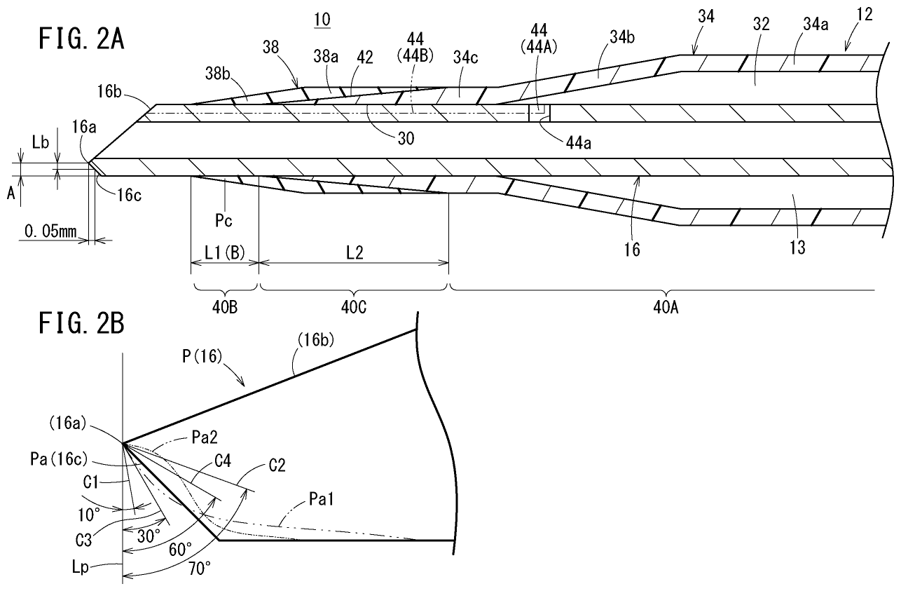

As illustrated to FIG. 2A, the catheter 12 has a close contact portion 30, which is in close contact with an outer circumferential surface of the inner needle 16 over the entire circumference, in at least a part of an inner circumferential surface. The close contact portion 30 is provided on the inner circumferential surface of a distal portion of the catheter 12. A flow path for flashback confirmation (hereinafter, referred to as “flashback flow path 32”) is formed between the catheter 12 and the inner needle 16 on the proximal side of the close contact portion 30. The flashback flow path 32 extends up to a proximal opening of the catheter 12.

The catheter 12 has a catheter body 34 that constitutes a main portion of the catheter 12 and a flexible portion 38 provided at a distal portion of the catheter body 34. Thus, the catheter 12 becomes more flexible toward the distal-most portion on the distal side. The flexible portion 38 is exposed from the housing 24 (FIG. 1).

The catheter body 34 accounts for most of the whole length of the catheter 12. Thus, the distal-most portion of the catheter body 34 is positioned near the distal-most end of the catheter 12. The catheter 12 and the flexible portion 38 are made of a resin material having flexibility. A creep strain of the catheter body 34 is greater than a creep strain of the flexible portion 38.

The catheter body 34 has: a straight portion 34a that has a constant outer diameter along the axial direction; a tapered portion 34b that extends from the straight portion 34a in the distal direction and has an outer diameter that decreases in the distal direction; and a distal constituting portion 34c that extends from the tapered portion 34b in the distal direction and constitutes a portion up to the distal-most portion of the catheter body 34. An inner circumferential surface of the distal constituting portion 34c is in close contact with (fitted to) the inner needle 16 in a liquid-tight manner over the entire outer circumferential surface.

The flashback flow path 32 is formed between an inner circumferential surface of the catheter body 34 (specifically, the straight portion 34a and the tapered portion 34b) and the outer circumferential surface of the inner needle 16. Among the catheter body 34 and the flexible portion 38, at least the catheter body 34 has transparency such that a flashback can be confirmed.

The catheter 12 is supported by the support member 26 (FIG. 1) at a spot of the catheter body 34 (the catheter body 34 is supported by the support member 26). As a result, it is possible to reliably support the catheter 12 and to reduce a sliding resistance at the time of advancing the catheter 12. Moreover, the portion supported by the support member 26 (FIG. 1) is located on the proximal side of an interface 42 between the catheter body 34 and the flexible portion 38, and thus, it is possible to prevent peeling of the interface 42 caused by sliding of the catheter 12 with respect to the support member 26.

It is preferably that the catheter body 34 be less likely to swell as compared with the flexible portion 38. As a result, it is possible to set an axial distance between a distal-most position of the inner needle 16 and a distal-most position of the catheter 12 to a desired size and to reduce a variation for each product during steam sterilization (autoclave sterilization) or ethylene oxide gas sterilization in a process of manufacturing the catheter assembly 10.

Examples of a constituent material of the catheter body 34 include a fluorine-based resin such as polytetrafluoroethylene (PTFE), an ethylene-tetrafluoroethylene copolymer (ETFE), and a perfluoroalkoxy fluorine resin (PFA), an olefin-based resin such as polyethylene and polypropylene or a mixture thereof, polyurethane, polyester, polyamide, a polyether nylon resin, a mixture of the olefin-based resin and an ethylene-vinyl acetate copolymer, and the like. A durometer hardness of the catheter body 34 is, for example, less than D70.

The flexible portion 38 includes the distal-most portion of the catheter 12. The flexible portion 38 is more flexible than the catheter body 34. That is, an elastic modulus k1 of the catheter body 34 and an elastic modulus k2 of the flexible portion 38 have a relationship of k1>k2.

The flexible portion 38 has: a straight portion 38a that has a constant outer diameter along the axial direction; and a tapered portion 38b that extends from the straight portion 38a in the distal direction and has an outer diameter decreasing in the distal direction. An inner circumferential surface of the flexible portion 38 is in close contact with (fitted to) an outer circumferential surface of the inner needle 16 in a liquid-tight manner over the whole outer circumference.

It is preferable that at least the flexible portion 38 between the catheter body 34 and the flexible portion 38 have an X-ray contrast property. As a result, for example, when the catheter 12 is broken in a blood vessel, it is possible to easily confirm a location of the catheter 12, which has been broken and left in the blood vessel by X-ray. A contrast layer in the case where the flexible portion 38 has the contrast property may be provided, for example, in any form of a stripe shape, an intermediate layer in the radial direction, or the whole layer.

Examples of a constituent material of the flexible portion 38 include various rubber materials such as natural rubber, butyl rubber, isoprene rubber, butadiene rubber, styrene-butadiene rubber, silicone rubber, various thermoplastic elastomers such as polyurethanes, polyesters, polyamides, olefins, and styrenes or a mixture thereof, and the like.

In the catheter 12, a single catheter body region 40A where only the catheter body 34, among the catheter body 34 and the flexible portion 38, exists, a single flexible portion region 40B where only the flexible portion 38, among the catheter body 34 and the flexible portion 38, exists, and a mixed region 40C where the catheter body 34 and the flexible portion 38 exist, are arranged in the axial direction. In the catheter 12 illustrated in FIG. 2A, the interface 42 between the catheter body 34 and the flexible portion 38 is formed in a tapered shape that is inclined at a substantially constant angle with respect to an axis of the catheter 12.

The single catheter body region 40A is a portion of the catheter body 34 present on the proximal side of a proximal-most portion of the flexible portion 38.

The single flexible portion region 40B is a portion of the flexible portion 38 present on the distal side of the distal-most portion of the catheter body 34. An axial length L1 of the single flexible portion region 40B is set to, for example, 0.3 to 5.0 mm, preferably 0.4 to 2.0 mm, and more preferably 0.5 to 0.9 mm. The hardness of the flexible portion (the single flexible portion region 40B) is, for example, A80 to D67 and preferably D53 to D64 at 23° C. The flexible portion 38 in the illustrated example is joined to the catheter body 34. Because the axial length L1 and the hardness of the single flexible portion region 40B are set within the above ranges, it is possible to prevent the distal end (the flexible portion 38) of the catheter 12 from being curled at the time of puncture. In addition, it is possible to preferably suppress catching by a blood vessel back wall 50a at the time of inserting the catheter 12. Further, it is possible to suppress crushing of the distal end of the catheter 12 at the time of suctioning blood.

The mixed region 40C is a portion in which the catheter body 34 and the flexible portion 38 are stacked in the radial direction. An axial length L2 of the mixed region 40C is set to, for example, 1 to 5 mm, and preferably 2 to 3 mm.

In the catheter 12 illustrated in FIG. 2A, the interface 42 between the catheter body 34 and the flexible portion 38 is inclined in the distal direction so as to approach the axis (center) of the catheter 12. Thus, the flexible portion 38 is present on the outer side of the catheter body 34 in the mixed region 40C.

The catheter assembly 10 may be provided with a needle protection member that covers the needle tip 16a when the inner needle 16 is removed from the catheter 12. In this case, a protrusion (not illustrated) is provided on the outer circumferential surface of the inner needle 16 to prevent the needle protection member from being removed from the inner needle 16 in the distal direction, and the protrusion is preferably provided on the proximal side of the single flexible portion region 40B. As a result, the interface 42 between the catheter body 34 and the flexible portion 38 is not caught by the protrusion at the time of advancing the catheter 12, and it is possible to prevent peeling of the interface 42 caused by the protrusion.

The catheter 12 is preferably coated seamlessly with a single coating material on the whole catheter 12 in order to eliminate (or minimize) a step at a boundary between the catheter body 34 and the flexible portion 38 on the inner circumferential surface and an outer circumferential surface of the catheter body 34.

Instead of the above configuration having the interface 42, the catheter 12 may be formed so as to become soft in the distal direction by changing each compounding amount of materials different in hardness in the axial direction. In this case, extrusion molding may be performed while changing each extrusion speed of different materials. Alternatively, a content of a plasticizer at the distal portion of the catheter 12 may be increased. In this case, the plasticizer may be applied to the distal portion of the catheter 12.

In the inner needle 16, a backcut portion 16c, which is continuous with the needle tip 16a and is inclined in a reverse direction to the blade face 16b with respect to the axis of the inner needle 16, is provided on the opposite side to a blade face 16b.

In order to prevent the inner surface of the catheter 12 from being pierced with the needle tip 16a when the catheter 12 is retracted after the catheter 12 is once moved forward with respect to the inner needle 16, the relationship between a height A of the backcut portion 16c and a length B from the distal-most portion of the catheter 12 to the distal-most portion of the catheter body 34 satisfies a formula of A>0.03B. In the present embodiment, the length B coincides with the length L1 of the single flexible portion region 40B described above. The height A of the backcut portion 16c is a radial length of the backcut portion 16c in the range from a distal-most portion to a proximal-most portion of the backcut portion 16c (total length of the backcut portion 16c).

In order to more effectively prevent the inner surface of the catheter 12 from being pierced with the needle tip 16a, the height (a radial length Lb) of the backcut portion 16c from the needle tip 16a at a position of 0.05 mm from the needle tip 16a in the proximal direction is set to, for example, 0.01 to 0.05 mm, and more preferably, set to 0.02 to 0.04 mm.

As illustrated in FIG. 2B, among projection lines P forming a contour shape of a projection image when the distal portion of the inner needle 16 is projected from just beside, at least a part of a projection line Pa, which corresponds to a portion (the backcut portion 16c in the present embodiment) of the distal portion of the inner needle 16 formed on the opposite side of the blade face 16b and continuous with the needle tip 16a, preferably passes between a straight line C1 (first straight line) at an angle of 10° with respect to a reference straight line Lp, which passes through the needle tip 16a and is perpendicular to a longitudinal direction of the inner needle 16, and a straight line C2 (second straight line) at an angle of 70° with respect to the reference straight line Lp. More preferably, at least a part of the projection line Pa passes between a straight line C3 (third straight line) at an angle of 30° with respect to the reference straight line Lp and a straight line C4 (fourth straight line) at an angle of 60° with respect to the reference straight line Lp. In FIG. 2B, the projection line Pa is a straight line, the whole projection line Pa passes between the straight line C1 and the straight line C2.

With this configuration, it is possible to achieve both the prevention of the pierce of the inner surface of the catheter 12 by the needle tip 16a and puncture properties with respect to the skin. When at least a part of the projection line Pa is present in a region closer to the reference straight line Lp than the straight line C1, the above-described pierce hardly occurs, but the puncture resistance becomes large. When at least a part of the projection line Pa is present in a region where an angle with the reference straight line Lp is larger than an angle with the straight line C2, the puncture resistance is low, but the above-described pierce is likely to occur.

The backcut portion 16c may be formed in a curved shape that bulges toward the reference straight line Lp like a projection line Pa1. At least a part of the projection line Pa1 preferably passes between the straight line C1 and the straight line C2, and more preferably passes between the straight line C3 and the straight line C4. The backcut portion 16c may be formed in a shape curved in an S shape like a projection line Pa2. At least a part of the projection line Pa2 preferably passes between the straight line C1 and the straight line C2, and more preferably passes between the straight line C3 and the straight line C4.

The inner needle 16 is provided with an introduction path 44 that communicates with the flashback flow path 32 to introduce blood into the flashback flow path 32. The introduction path 44 illustrated in FIG. 2A is a side hole 44A which penetrates through a wall portion of the inner needle 16 in the radial direction. As illustrated by an imaginary line in FIG. 2A, the introduction path 44 may be a groove portion 44B extending in the axial direction on the outer circumferential surface of the inner needle 16.

In the initial state of the catheter assembly 10 illustrated in FIG. 2A, a proximal end 44a of the introduction path 44 is provided on the proximal side of an axial center position Pc of the single flexible portion region 40B. More specifically, at least the proximal end 44a of the introduction path 44 is provided on the proximal side of a distal-most portion of the single catheter body region 40A. The entire side hole 44A is provided on the proximal side of the distal-most portion of the single catheter body region 40A. Incidentally, a part of the side hole 44A may be present on the distal side of the distal-most portion of the single catheter body region 40A.

The entire side hole 44A is provided on the proximal side of the mixed region 40C. A part of the side hole 44A may be present on the distal side of a proximal end of the mixed region 40C.

Regarding a position of the introduction path 44 in relation to the close contact portion 30, at least the proximal end 44a of the introduction path 44 (the side hole 44A or the groove portion 44B) is provided on the proximal side of the close contact portion 30 in the initial state of the catheter assembly 10 illustrated in FIG. 2A. The entire side hole 44A is provided on the proximal side of the close contact portion 30.

Next, functions of the catheter assembly 10 configured as described above will be described.

In use of the catheter assembly 10 illustrated in FIG. 1, a puncturing operation to puncture the patient's skin with the catheter assembly 10 is performed. In the puncturing operation, a user (a doctor, a nurse, or the like) presses the distal portion of the catheter assembly 10 against the patient while gripping the housing 24, thereby puncturing the skin toward a puncture target blood vessel. Accordingly, the skin is punctured with the inner needle 16 and each distal portion of the catheter 12.

Next, the user operates the catheter operation member 20 in the distal direction to cause the catheter member 17 (the catheter 12 and the catheter hub 14) to advance while fixing the position of the needle hub 18 (the housing 24). Accordingly, the catheter 12 is inserted to the target position in the blood vessel.

Next, the user pulls the housing 24 in the proximal direction while holding the positions of the catheter operation member 20 and the catheter member 17. Accordingly, the catheter member 17 and the catheter operation member 20 completely come out of the housing 24, and the inner needle 16 is removed from the catheter 12 in the proximal direction.

Next, the catheter operation member 20 is detached from the catheter hub 14. Accordingly, the catheter member 17 is indwelled in the patient. Incidentally, the catheter operation member 20 may be kept attached to the catheter hub 14 depending on a preference of the user.

Next, the connector of the infusion tube (not illustrated) is connected to the proximal side (the proximal portion of the catheter hub 14) of the catheter member 17 from which the inner needle 16 has been removed, and the infusion solution (medicinal liquid) is administered from the infusion tube to the patient.

In this case, the catheter assembly 10 according to the present embodiment has the following effects.

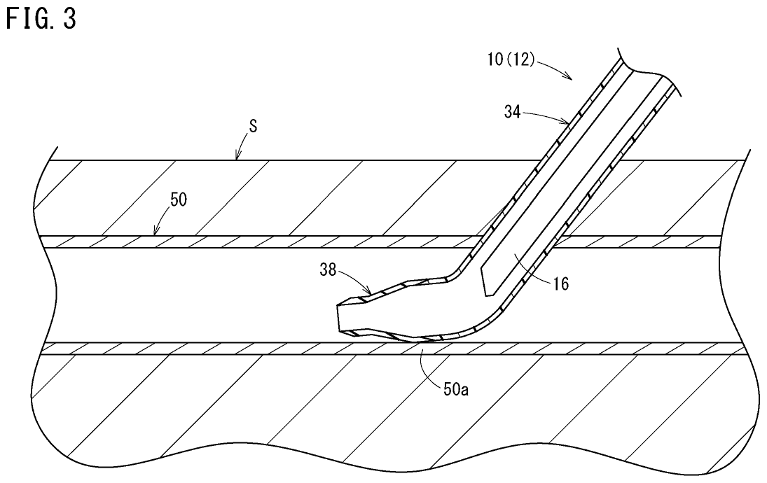

According to the catheter assembly 10, the flexible portion 38, which is more flexible than the catheter body 34, is provided at the distal portion of the catheter body 34 as illustrated in FIG. 2A. Thus, it is possible to prevent the distal end of the catheter 12 from being caught by a blood vessel back wall 50a, which is a blood vessel wall of the blood vessel 50 on the opposite side of a puncture spot at the time of advancing the catheter 12 to insert the catheter 12 into the blood vessel 50 after puncturing a skin S with the distal portion of the catheter assembly 10 even when a puncture angle, which is an angle between a central axis of the blood vessel 50 to be punctured and a central axis of the inner needle 16 with which the puncture is performed, is large as illustrated in FIG. 3.

That is, the flexible portion 38 is brought into contact with the blood vessel back wall 50a and is pressed by the blood vessel back wall 50a to be easily deformed at the time of advancing the catheter 12 as illustrated in FIG. 3, and thus, it is possible to prevent the distal end of catheter 12 from being caught by the blood vessel back wall 50a. As a result, it is possible to prevent the catheter 12 from being hardly inserted into the blood vessel 50 or to prevent the blood vessel back wall 50a from being damaged by the distal end of the catheter 12.

In addition, as illustrated in FIG. 2A, the relationship between the height A of the backcut portion 16c and the length B from the distal-most portion of the catheter 12 to the distal-most portion of the catheter body 34 satisfies the formula of A>0.03B. With this configuration, the height A of the backcut portion 16c is appropriately high, and thus, it is possible to inhibit the inner surface of the catheter 12 from being pierced with the needle tip 16a. When the catheter is retracted in a state in which the distal end of the catheter is deformed toward the blade face, piercing with the needle tip of the inner needle can occur. Therefore, a length and a height of each site are considered in order to inhibit the occurrence of piercing. Because the amount of deformation of the distal end of the catheter 12 toward the blade face 16b depends on the length B from the distal-most portion of the catheter 12 to the distal-most portion of the catheter body 34, the height A of the backcut portion 16c also needs to be high when the length B is long. The preferred relationship between the length B and the height A in the embodiment is the above formula.

The flashback flow path 32 is formed between the catheter 12 and the inner needle 16, and the inner needle 16 is provided with the introduction path 44 that communicates with the flashback flow path 32 to introduce blood into the flashback flow path 32. The proximal end 44a of the introduction path 44 is provided on the proximal side of the axial center position Pc of the portion of the flexible portion 38 present on the distal side of the distal-most portion of the catheter body 34. In addition, the catheter 12 has the close contact portion 30 in which at least a part of the inner circumferential surface is in close contact with the outer circumferential surface of the inner needle 16, and at least the proximal end 44a of the introduction path 44 is provided on the proximal side of the close contact portion 30.

With the above configuration, it is possible to prevent the flexible portion 38 from being deformed to block the introduction path 44 at the time of puncture, and thus, it is possible to easily confirm the flashback of blood.

In the close contact portion 30, both the flexible portion 38 and the catheter body 34 are in close contact with the inner needle 16. With this configuration, an appropriate fitting force between the inner needle 16 and the catheter 12 can be obtained. With the appropriate fitting force, the flexible portion 38 is prevented from being curled, and the inner needle 16 can be easily removed from the catheter 12 at the time of puncturing the skin S.

The catheter 12 has the mixed region 40C in which the catheter body 34 and the flexible portion 38 overlap each other in the radial direction. With this configuration, a change in rigidity from the catheter body 34 to the flexible portion 38 can be made gradual, and thus, it is possible to more favorably inhibit the distal end of the catheter 12 from being caught by the blood vessel back wall 50a at the time of inserting the catheter 12 into the blood vessel 50.

The axial length L1 of the single flexible portion region 40B (the portion of the flexible portion 38 present on the distal side of the distal-most portion of the catheter body 34) is 0.3 to 5.0 mm. With this configuration, it is possible to suppress the curling of the distal end (the flexible portion 38) of the catheter 12 at the time of puncture. In addition, it is possible to more preferably suppress the catching by the blood vessel back wall 50a at the time of inserting the catheter 12. Further, it is possible to suppress crushing of the distal end of the catheter 12 at the time of suctioning blood.

The present invention is not limited to the above-described embodiment, and various modifications can be made within a scope not departing from a gist of the present invention.

What is claimed is:

1. A catheter assembly comprising:
a catheter; and
an inner needle inserted through the catheter and comprising a needle tip located at a distal-most end of the inner needle, wherein:
the catheter comprises:
a catheter body, and
a flexible portion that is located at a distal end of the catheter body, includes a distal-most portion of the catheter, and is more flexible than the catheter body,
the inner needle comprises a backcut portion that is continuous with the needle tip and is inclined in a reverse direction to the blade face with respect to an axis of the inner needle, and
a relationship between (i) a height A of the backcut portion in a direction perpendicular to the axis of the inner needle, and (ii) a length B from the distal-most portion of the catheter to a distal-most portion of the catheter body satisfies a formula A>0.03B.

2. The catheter assembly according to claim 1, wherein:
a height of the backcut portion from a needle tip, in the direction perpendicular to the axis of the inner needle, at a position 0.05 mm proximal direction from the needle tip, is in a range of 0.01 to 0.05 mm.

3. The catheter assembly according to claim 1, wherein:
among projection lines that form a contour shape of a projection image when a distal portion of the inner needle is projected from a side, at least a part of a projection line corresponding to the backcut portion passes between (i) a first straight line forming an angle of 10° with a reference straight line that passes through the needle tip and is perpendicular to a longitudinal direction of the inner needle, and (ii) a second straight line forming an angle of 70° with the reference straight line.

4. The catheter assembly according to claim 3, wherein:
at least the part of the projection line corresponding to the backcut portion passes between (i) a third straight line forming an angle of 30° with the reference straight line, and (ii) a fourth straight line forming an angle of 60° with the reference straight line.

5. The catheter assembly according to claim 1, wherein:
a flow path for flashback confirmation is formed between the catheter and the inner needle,
the inner needle comprises an introduction path that communicates with the flow path to introduce blood into the flow path, and
a proximal end of the introduction path is located on a proximal side of an axial center position of a portion of the flexible portion present on a distal side of a distal-most portion of the catheter body.

6. The catheter assembly according to claim 1, wherein:
the catheter has a close contact portion where at least a part of an inner circumferential surface of the catheter is in close contact with an outer circumferential surface of the inner needle, and
both the flexible portion and the catheter body are in close contact with the inner needle at the close contact portion.

7. An inner needle configured to be inserted through a catheter that comprises a catheter body, and a flexible portion that is located at a distal end of the catheter body, includes a distal-most portion of the catheter, and is more flexible than the catheter body, the inner needle comprising: comprising:
a needle tip located at a distal-most end of the inner needle, and
a backcut portion that is continuous with the needle tip and is inclined in a reverse direction to the blade face with respect to an axis of the inner needle,
wherein a relationship between (i) a height A of the backcut portion in a direction perpendicular to the axis of the inner needle, and (ii) a length B from the distal-most portion of the catheter to a distal-most portion of the catheter body satisfies a formula A>0.03B.

8. The inner needle according to claim 7, wherein:
a height of the backcut portion from a needle tip, in the direction perpendicular to the axis of the inner needle, at a position 0.05 mm proximal direction from the needle tip, is in a range of 0.01 to 0.05 mm.

9. The inner needle according to claim 7, wherein:
among projection lines that form a contour shape of a projection image when a distal portion of the inner needle is projected from a side, at least a part of a projection line corresponding to the backcut portion passes between (i) a first straight line forming an angle of 10° with a reference straight line that passes through the needle tip and is perpendicular to a longitudinal direction of the inner needle, and (ii) a second straight line forming an angle of 70° with the reference straight line.

10. The inner needle according to claim 9, wherein:
at least the part of the projection line corresponding to the backcut portion passes between (i) a third straight line forming an angle of 30° with the reference straight line, and (ii) a fourth straight line forming an angle of 60° with the reference straight line.
